(19) United States
(12) Patent Application Publication
(10) Pub. No.: US 2017/0044483 A1
(43) Pub. Date: Feb. 16, 2017

(54) Method and Apparatus for Purifying Biological Molecules

(71) Applicant: Robert Bosch GmbH, Stuttgart (DE)

(72) Inventors: Bernd Faltin, Gerlingen (DE); Christian Dorrer, Winnenden (DE)

(73) Assignee: Robert Bosch GmbH, Stuttgart (DE)

(21) Appl. No.: 15/305,172

(22) PCT Filed: Apr. 17, 2015
(86) PCT No.: PCT/EP2015/058348

§ 371 Date: Oct. 19, 2016

(30) Foreign Application Priority Data
Apr. 25, 2014 (DE) 102014207774.5

(51) Int. Cl. C12M 1/00; C12N 1/06

(52) U.S. Cl. CPC C12M 47/06; C12M 47/10; C12N 1/06

(57) ABSTRACT

An apparatus for purifying biological molecules, especially nucleic acids or proteins, includes at least one filter and a circuit. A method of purifying biological molecules using the apparatus includes pumping at least some fluids into the circuit via the filter. Fluid with biological cells is pumped into the circuit via the filter, and cells retained on the filter are digested. Binding buffer is pumped into the circuit via the filter in order to bind the biological molecules to the filter. A washing buffer is pumped into the circuit via the filter in order to clean the biological molecules bound to the filter so that the biological molecules bound to the filter are enabled for further use.

The present invention relates to a method and a device for purifying biological molecules, more particularly nucleic acids or proteins, the method using at least one filter.

### PRIOR ART

There are many different methods for purifying biological molecules and, more particularly, nucleic acids or proteins. Generally, the biological molecules are obtained from cell material, i.e., from prokaryotic or eukaryotic cells. Before intracellular material can be processed further, it is generally necessary to disrupt the cells themselves. This cell disruption is generally also referred to as cell lysis. After removal of the cell debris, it is, for example, possible for nucleic acids, protein or peptides to be purified, processed and analyzed further. When proteins are mentioned hereinafter, this also means peptides. In order, for example, to be able to detect a particular nucleic acid, the purified nucleic acids can be selectively amplified by means of a PCR (polymerase chain reaction), making it possible to detect the particular nucleic acid sequence.

The disruption of the cells can be achieved in different ways. An enzymatic disruption of the cells is common, involving, for example, carrying out a treatment with the enzymes proteinase K or lysozyme. A thermal cell disruption by heating and/or freezing of the sample or a cell disruption using chemical reagents is possible too. Furthermore, the cell disruption can be achieved mechanically, for example by means of an ultrasound treatment. A customary method for the further purification of, for example, nucleic acids envisages the so-called lysate which arises as a result of the cell disruption being admixed with a binding buffer and being contacted with a solid matrix, for example a silica filter or a silica membrane. In this case, the nucleic acids adsorb to the filter and can be subsequently washed with a wash buffer and then eluted from the solid matrix and used further. Various commercially available kits and laboratory instruments work according to this principle.

In many cases, it is necessary to concentrate the cell material prior to further treatment or to accumulate the cells. To this end, a centrifugation of the sample containing the cells can be carried out for example. German published specification DE 10 2005 009 479 A1 describes a method in which the cells are accumulated by means of a filtration. Using such a filter membrane method, it is, for example, also possible to quantify the accumulated cells, for example bacteria, as described in the publication by Dufour, Alfred P., et al. (Applied and Environmental Microbiology, May 1981, pages 1152-1158).

German published specification DE 10 2010 030 962 A1 describes a method for hybridizing nucleic acids in a microarray, in which the sample is pumped first through a denaturation unit and then through a separate reaction zone containing the microarray with the immobilized probes. In this case, the pump route can be designed as a circuit.

German published specification DE 10 2010 043 015 A1 discloses a method in which nucleic acids are amplified, i.e., reproduced, on a filter. It is possible to carry out beforehand a concentration and a lysis of the cells containing the nucleic acids on the filter.

### DISCLOSURE OF THE INVENTION

### Advantages of the Invention

Using the method according to the invention, it is possible to concentrate and purify biological molecules, more particularly nucleic acids or proteins or other biological molecules. The purification is achieved in principle by a nonspecific adsorption of the biological molecules to a matrix, more particularly to a membrane. Hereinafter, a filter will generally be mentioned, and what is meant here is the matrix especially in the form of a membrane or, for example, in the form of a packed bed. In this connection, the core of the invention is that at least some of the liquids required for a process effectuation are pumped in a circuit across the filter. Specifically, the steps of the method according to the invention comprise, first of all, the pumping of a liquid containing biological cells, i.e., a sample liquid, across the filter. The term “biological cells” are to be generally understood to mean cells from which biological molecules, such as nucleic acids or proteins for example, are to be prepared or purified. They can be, for example, pathogenic microorganisms such as bacteria or fungi. However, the method according to the invention is also suitable for human cells or other cells and can generally be used for the purification of proteins or nucleic acids from prokaryotic or eukaryotic cells. The term “sample liquid” is to be generally understood to mean the liquid containing the corresponding cells, for example a cell suspension or a patient sample, for example blood, lavage fluid, urine, cerebrospinal fluid, sputum or a rinsed swab or smear. Depending on the application, the volume of the sample may vary, for example between a few μl to 10 ml. After sample application, the cells retained on the filter are disrupted, it being possible in principle to use various methods for the cell disruption. The biological molecules present in the cell lysate are bound to the filter by means of a binding buffer, the binding buffer being pumped in a circuit across the filter. The biological molecules bound to the filter are cleaned with wash buffer in the following step, which wash buffer is pumped across the filter. As a result, the biological molecules to be purified are then present in reversibly immobilized form on the filter. For further processing or analysis, the bound biological molecules can be eluted from the filter in the customary way or the filter containing the reversibly immobilized biological molecules is further used as such directly.

In methods known to date, the accumulation of cells from a sample is achieved by centrifugation. However, implementation of this method in a microfluidic system and, hence, cost-effective automation is not possible. Methods are also known in which the accumulation of the cells from a sample is achieved by flushing across a filter, the lysis then taking place by application of a lysis buffer to the filter. However, in the case of integration into a microfluidic system, said methods have the disadvantage that the exact positioning of the lysis buffer on the filter is difficult or associated with great effort. Said positioning must be done manually, or cameras or light barriers, for example, are required. Furthermore, when applying the lysis buffer to the filter, there is the risk of displacing cells and already released nucleic acids from the filter, which are then no longer available for further purification and thus lower the efficiency of the purification. Furthermore, in said methods, the diffusion of lysis reagents, for example enzymes, on the filter is hampered, lowering the effectiveness of the lysis, especially in the case of difficult-to-lyse cells, for example fungi. The invention solves these problems and thereby allows the simple realization of the described method in an automated microfluidic system (lab-on-chip system).

Owing to the circular guidance of the liquids, especially during the process of binding the biological molecules to the filter, the substances are flushed repeatedly through the filter, and this achieves the particular effectiveness of the purification method according to the invention. As a result of this guidance of the liquids in a circular fluidic path, the filter material is contacted repeatedly with the liquids. In the course of this, a saturation equilibrium ensues, in which the maximum binding capacity of the membrane is exhausted. In the case of conventional methods, it is often the case that only a portion of the molecules of interest is actually adsorbed. In the case of the method according to the invention, the circular fluid guidance ensures that, for example, all nucleic acids which were released during the cell disruption are pumped effectively across the filter in the binding step. Virtually no nucleic acids are lost, increasing the yield. Furthermore, the pumping in a circuit ensures that an optimal mixing of the reagents, i.e., of the binding buffer and of the cell lysate for example, takes place. The mixing of reagents is frequently a problem, especially in microfluidic systems. The circular fluid guidance envisaged according to the invention ensures a good mixing of the various reagents and buffers, and, as a result, the method according to the invention is especially advantageously suitable for realization within a microfluidic system. Independent thereof, the efficiency of mixing especially in a microfluidic system can also be additionally increased by further measures, more particularly by mixer structures or mixing chambers known per se, in a microfluidic system.

In the first method step, there is an accumulation of the biological cells, the cells being retained on the filter according to the size-exclusion method and/or by electrostatic interactions when the sample liquid is pumped across the filter. The more sample liquid that is pumped across the filter, the higher the number of accumulated cells. The diameter of such a filter known per se may vary, for example between 1 and 25 mm, depending on the dimension of the device according to the invention. Suitable filters are, for example, fiber filters, fabric filters and/or membrane filters, especially composed of silica. Furthermore, bead beds, more particularly microbead beds, for example composed of silica beads, are also suitable. The pore diameter of the materials is preferably below 100 μm.

When applying the sample liquid to the filter, it can be envisaged that the sample is pumped in a circuit repeatedly across the filter. This has the advantage that even cells which may not have been retained during the first filter passage are retained during a repeated filter passage. Since electrostatic forces are also acting and the size distribution of the pores in a silica filter cannot generally be assumed to be homogeneous, there is the possibility of cells not being retained during a first passage. Furthermore, cells can be caught in “dead ends” of the system, meaning that effectiveness can be increased by means of a circular guidance during sample application.

The disruption of the cells can be achieved in various ways, for example by mechanical means or by means of heat. What can be advantageously envisaged, for example, is an ultrasound treatment, which can be carried out with comparatively low expenditure in terms of apparatus. In this case, there is no need for additional reagents for the lysis or the cell disruption. The ultrasound can be directly inputted into the filter. To this end, it is, for example, possible to realize the wall of a corresponding filter chamber as a membrane into which the ultrasound is coupled in by means of a horn. During the ultrasound treatment, the filter chamber should be filled with liquid or with a buffer or water. The ultrasound treatment may lead to the filter material being partly broken up. Firstly, this at least partly releases the cells accumulated in the filter and makes them accessible to the lysis action due to the ultrasound. Secondly, the particles arising here can generate an additional grinding action and thereby further support the cell disruption, the particles being collected by the intact regions of the filter later on in processing.

Particular preference is given to a cell disruption using enzymes or other lysing reagents, for example chemical reagents. In the case of this cell disruption with lysing reagents, a circuit guidance of the liquids can likewise be particularly advantageously envisaged. To this end, an appropriate lysis buffer is fed into the circular fluidic path and pumped in a circuit across the filter. In this case, pumping is carried out especially in the direction in which the sample was also pumped across the filter. This avoids loss of cells or already released nucleic acids during the initial application of the lysis buffer to the filter. Furthermore, air bubbles, which may reach the filter, are removed therefrom later on. In the case of the methods known to date, such air bubbles remain on the filter and suppress the lysis locally. Furthermore, by continuously bringing lysis buffer to the cells, an impoverishment of the lysis reagents on the filter is avoided and thus a particularly effective and complete lysis of the cells on the filter is achieved. It has become apparent that, surprisingly, when using enzymes for the lysis, their activity is not lowered by the constant pumping and the resultant shear forces, making it advantageously possible to carry out even an enzymatic lysis according to the scheme described. Depending on the type of biological cells and of reagents used, the lysis may, for example, require a period between 2 and 30 minutes. A lysis buffer is to be understood to mean a buffer of a kind which is suitable for the cell disruption or for the lysis of the target cells. The buffer can, for example, contain lysis enzymes known per se, such as lysozyme and/or proteinases for example. Alternatively or additionally, chaotropic salts, detergents and/or basic ingredients such as, for example, NaOH can be present. Furthermore, buffer substances (e.g., Tris-HCl), nuclease inhibitors (e.g., EDTA or EGTA) and/or reducing agents (e.g., β-mercaptoethanol) can be present.

In the following binding step, the binding buffer is fed into the circular fluidic path and pumped in a circuit. In this case, the binding buffer mixes with the lysate and, for example, the nucleic acids bind to the filter under the conditions thereby set.

Prior to the binding step, a further denaturation step can be carried out, especially when purifying nucleic acids. A buffer suitable for this purpose can contain chaotropic reagents in particular, for example GIT (guanidinium isothiocyanate). By means of such reagents, the proteins present in the lysate are “salted” so to speak, making it easier to wash out the proteins. The denaturation buffer is preferably likewise pumped in a circuit in order to further increase the effectiveness of the denaturation step.

Prior to the binding step, an additional digestion step can be carried out, especially when purifying nucleic acids. An appropriate digestion buffer can, for example, contain various enzymes, more particularly proteinases, which bring about a digestion of the proteins released in the lysis step. This can further improve the effectiveness of a nucleic acid purification and the purity of the nucleic acids obtained. In the case of this step, the appropriate digestion buffer is fed into the path and likewise advantageously pumped in a circuit. Here too, it has become apparent that, surprisingly, the activity of the enzymes used for the digestion is not lowered by the constant pumping and the resultant shear forces.

After the binding of the biological molecules to the filter, one or more wash steps are carried out, with wash buffer being conducted across the filter. The composition of appropriate wash buffers is selected such that, for example, the nucleic acids remain bound to the filter in this step, whereas other molecules, more particularly proteins, are not adsorbed and removed. An alcohol-containing wash buffer, for example 70% EtOH, can, for example, be used as wash buffer.

For many applications, it is advantageous when the filter is dried after the treatment with wash buffer. This can, for example, be achieved by passing air or nitrogen across the filter. Thereafter, an elution of the adsorbed target molecules from the filter can take place, it being possible to use water or an appropriate elution buffer for this purpose. It can also be envisaged that the filter containing the target molecules adsorbed thereto is further used as such. For example, it is possible to carry out a PCR using the nucleic acids reversibly immobilized on the filter, as is known per se from the prior art.

When using certain samples, it may be advantageous to pretreat the sample prior to application to the filter. For example, when filtering blood, there may be the problem of the filter being clogged and thus blocked with blood cells, making a further filtration impossible. To this end, it has been found to be advantageous to first selectively lyse the blood cells. Here, “lyse selectively” means that the blood cells, which are also referred to as human cells, are disrupted, whereas other cells present in the sample, more particularly pathogens, remain intact. This can, for example, be achieved by the treatment of the sample with chaotropic reagents or detergents or by osmotic shock and has the advantage that a clogging of the filter is avoided. In the case of such a selective lysis, a commercially available kit (Molzym MolYsis Complete5) additionally carries out a digestion of the released human nucleic acids by means of a DNase. Such a digestion can be integrated into the method according to the invention and have the advantage that the filterability of the sample is further improved and a human nucleic acid background is removed in part from the sample. For example, the sample is first mixed with a chaotropic buffer and then incubated with a DNase, for example for a period of 10 min, before the sample is applied to the filter.

In a preferred embodiment of the method according to the invention, one or more of the method steps can at least in part be carried out with input of heat. Especially for the cell disruption and/or the additional digestion step and/or for the drying of the filter, it may be advantageous to increase the temperature. For example, the enzymes used for an enzymatic cell disruption may have an elevated temperature optimum, and so the lysis of the cells can proceed more rapidly and effectively in the case of a temperature increase, for example to temperatures between 35 and 60° Celsius, more particularly between 35 and 45° Celsius. The same applies, mutatis mutandis, to a digestion or denaturation step. The drying of the filter can, too, be quickened by a temperature increase, for example by a temperature increase to a temperature between 40 and 60°. In general, the filter in particular can be directly heated for the input of heat, for example via a Peltier element known per se or a film heater which is contacted with the unit containing the filter. Furthermore, it is also possible to work with temperature-adjusted liquids; for example, it can be envisaged to heat at least in part a prestorage vessel for the liquids used and/or the conduit system. Depending on the application, a cooling or, in general, an adjustment of temperature may also be advantageous.

In a particularly preferred embodiment of the method according to the invention, the pumping direction in one or more of the method steps can be reversed once or repeatedly. As a result, particularly a blockage of the filter or a clogging of the filter can be avoided or possibly undone. Furthermore, the mixing of liquids present in the circuit can be improved and precipitated solids may possibly be brought back into solution. The reversal of the pumping direction after binding of the target molecules to the filter has taken place does not generally lead to a detachment of the target molecules from the filter, since the adsorption of the molecules to the filter is independent of the pumping direction. A reversal of the pumping direction is advantageous especially during the binding step and/or during the wash step and/or during the elution step. Even during the sample application or during the accumulation of the cells on the filter, it may be advantageous to briefly repeatedly reverse the pumping direction in order to avoid a clogging of the filter with cells and thus a blockage or to possibly undo them.

The method according to the invention is particularly advantageously carried out in a microfluidic device. In general, microfluidic devices have the advantage that they are especially suitable for automated processes. Analysis duration and costs and the risk of contamination are reduced as a result of an automation. Furthermore, an automated system does not necessarily need to be operated by skilled personnel, since operation is generally easy to learn. The method according to the invention in cooperation with a microfluidic device offers the particular advantage that an especially good mixing of the liquids is achieved owing to the circular guidance of the liquids. In many cases, it is therefore possible to dispense with further structures and active components such as stirrers for a mixing. Nevertheless, it is, however, also possible to envisage additional mixer structures or mixing chambers known per se in a corresponding device in order to further increase the efficiency of mixing.

The invention further comprises a device for carrying out a purification of biological molecules, more particularly of nucleic acids or proteins. The device has at least one pump for pumping liquids. Furthermore, the device comprises at least one unit for fixing at least one filter. The purification protocols which can be carried out therewith are based on the biological molecules to be purified being able to adsorb to the filter. According to the invention, the device has a conduit system for the circular pumping of liquids across the filter. Especially the described method according to the invention can be advantageously carried out using this device. In this connection, an essential aspect of the invention is that the efficiency of the purification method can be substantially improved by the circular pumping of liquids across the filter.

The unit for fixing the filter is especially a filter chamber for accommodating the filter material. The expression “filter chamber” is to be understood to mean especially a fluidic cavity containing a filter. The filter chamber can, for example, be designed as a tube or, particularly preferably, as a microfluidic element. The filter chamber preferably has a multilayer structure. In this case, two or more structured plates, more particularly polymer plates, can be envisaged for example. In one of the plates, a planar recess can be envisaged, into which the filter, for example a membrane, or some other filter material, for example a microbead bed, can be inserted. In this case, it has been found to be advantageous, especially in the case of comparatively large filter diameters (>3 mm), to support the filter by means of a support structure, for example a porous polymer support (frit), in order to avoid a deflection or sagging of the filter. One or more inlet and outlet channels are envisaged for the feed-through of liquids. Furthermore, it can be envisaged that an additional membrane is inserted between the two plates, by means of which membrane it is possible to realize additional functionalities of the filter chamber, for example a pneumatic operation of membrane valves and/or a membrane pump. Preferably, cover films or lidding membranes or other polymer layers are envisaged as lateral external borders of the system. Furthermore, a cover film can be utilized for coupling in ultrasound. Especially in embodiments of the device which are geared to an ultrasound treatment in the cell lysis, it can be envisaged that the filter chamber has an expansion for introducing the ultrasound. In this case, the expansion, which can be circular or part-circular for example, is appropriately outwardly realized as an aperture which can be closed especially with a lidding membrane. Furthermore, said expansion of the filter chamber can take on the function of a venting means for the system, it being possible to envisage venting channels which open into the expansion.

In a particularly preferred embodiment, the device according to the invention has at least one vented vessel by means of which liquid can be introduced into the system. More particularly, an upwardly vented vessel can be fitted into the fluidic path or into the conduit system for the circular pumping of liquids across the filter. The fitting of a vented vessel has the advantage that the increasing volume of the liquids present in the circuit can be gathered and the pressure in the circuit can be kept constant, and, as a result, a microfluidic realization is particularly advantageously possible. The liquids can, for example, be introduced into the vessel by hand, more particularly by pipette, or by pumping with a second pump integrated into the system. Furthermore, a vented vessel has the advantage that air bubbles which have entered the fluidic path can rise upward in the vessel and thus leave the system. For instance, it is avoided that air bubbles remain in the circuit and lead to foam formation. Such a vessel can, for example, be realized as a tube, chamber or as some other fluidic element which has a volume between 100 μl and 10 ml for example. Furthermore, multiple vented vessels can be particularly advantageously envisaged in the system, which vessels can serve especially as prestorage chambers for reagents.

The pump can, for example, be a peristaltic pump or a micromembrane pump. Particularly advantageously, the pump is more or less directly upstream or downstream of the filter, making it possible to pump the liquids with very high positive or negative pressure across the filter. In this connection, the channel piece between the pump and the filter is appropriately comparatively short. Furthermore, it can be advantageously envisaged that an inlet channel for the sample liquids opens more or less directly in front of the pump or the filter.

This has the advantage that other prestorage vessels for buffer solutions are not contaminated by the sample liquid. It can also be envisaged that multiple inlet channels are provided for various liquids.

Particularly advantageously, one or more elements of the device can be heatable. For example, the pump and/or the conduit system or parts thereof and/or the filter and/or possibly a vessel which is envisaged for the prestorage or for the introduction of liquids can be heatable. In this way, it is possible to carry out individual method steps with adjustment of temperature. For example, the lysis step can be carried out at an elevated temperature by the lysis buffer being prewarmed and/or the filter itself being heated.

Preferably, the device according to the invention is designed as a microfluidic system. With regard to the advantages of the device according to the invention in a microfluidic design, reference is made to the advantages already mentioned above. The method according to the invention and the device according to the invention can, for example, be particularly advantageously implemented in molecular diagnostics and/or, for example, in a lab-on-a-chip system.

Further features and advantages of the invention are revealed by the following description of exemplary embodiments in conjunction with the drawings. Here, the individual features can each be realized separately or in combination with one another.

### BRIEF DESCRIPTION OF THE DRAWINGS

The drawings show:

FIG. 1 schematic depiction of the principle behind the circular fluid guidance;

FIG. 2 schematic depiction of the components of an exemplary embodiment of a device for carrying out the method according to the invention;

FIG. 3 schematic depiction of a further exemplary embodiment of a device for carrying out the method according to the invention;

FIG. 4 schematic depiction of a further exemplary embodiment of a device for carrying out the method according to the invention;

FIG. 5 schematic depiction of a further exemplary embodiment of a device for carrying out the method according to the invention;

FIG. 6 schematic depiction of a multilayer filter chamber as a constituent of the device according to the invention;

FIG. 7 microfluidic device according to the invention in top view;

FIG. 8 lateral view of the multilayer structure of the microfluidic device from FIG. 7;

FIG. 9 diagonal view of the microfluidic device from FIG. 7;

FIG. 10/11 detailed views of an exemplary filter chamber of a microfluidic device according to the invention in side view (FIG. 10) and in top view (FIG. 11) and

FIG. 12/13 detailed view of a further exemplary filter chamber of a microfluidic device according to the invention in side view (FIG. 12) and in top view (FIG. 13).

### DESCRIPTION OF EXEMPLARY EMBODIMENTS

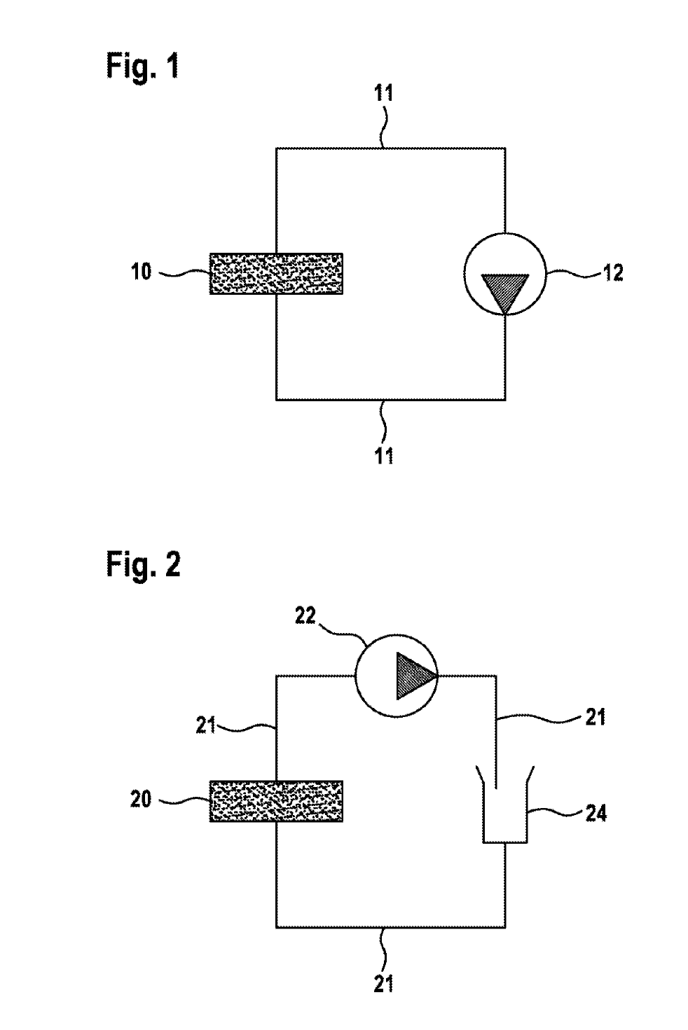

The schematic depiction in FIG. 1 illustrates the principle behind a circular fluidic path 11, which runs across a filter 10. The liquid in the fluidic connections 11 is driven via a pump 12. The fluidic connections 11 can, for example, be formed by pieces of tubing or by channels. The pump 12 is a liquid pump, for example a peristaltic pump or a membrane pump. In the case of a microfluidic design of the device, it is possible to use customary integratable microfluidic pumps. The filter 10 depicted here is realized in the form of a filter chamber. The hereinafter described depiction of a filter is to be understood in many cases as a synonym for a filter chamber. The filter chamber can be designed as a microfluidic component. The nature of the flow to and from the filter 10 can optionally be precisely set. The filter chamber itself can, for example, be realized in a multilayer structure composed of multiple structured polymer layers. Owing to this way of construction, an especially cost-effective manufacture is possible. When the pump 12 is connected to the inlet and outlet of the filter chamber 10 via pieces of tubing, feeding into the fluidic path can, for example, take place by opening of the hose connection and pipetting.

FIG. 2 shows a preferred variant for the schematic structure of a device for carrying out the method. In addition to the filter or the filter chamber 20 and the pump 22 and the fluidic connections 21, an upwardly vented vessel 24 is integrated into the fluidic path. Via said vessel 24, it is possible to introduce liquids into the circular fluidic path. The necessary solutions or buffers can, for example, be pipetted into the vessel 24. As a result, it is advantageously possible, during the process, to feed buffers or other solutions into the fluidic path. Furthermore, this design offers the advantage that air bubbles which have entered the fluidic path can rise upward in the vessel 24 and thus leave the system. The volume of the vessel 24 can be appropriately selected depending on the design of the arrangement, for example between 100 μl and 10 ml. The vessel 24 can, for example, be realized as a tube or as a microfluidic element. Advantageously, the outlet channel of the system is situated on the bottom side of the vessel 24. Here, “bottom side” means the part of the vessel which is situated at the lowest point with regard to gravity. This has the advantage that liquids can be completely removed from the vessel. Advantageously, the vessel 24 is in this case designed such that it becomes narrower in a downward direction.

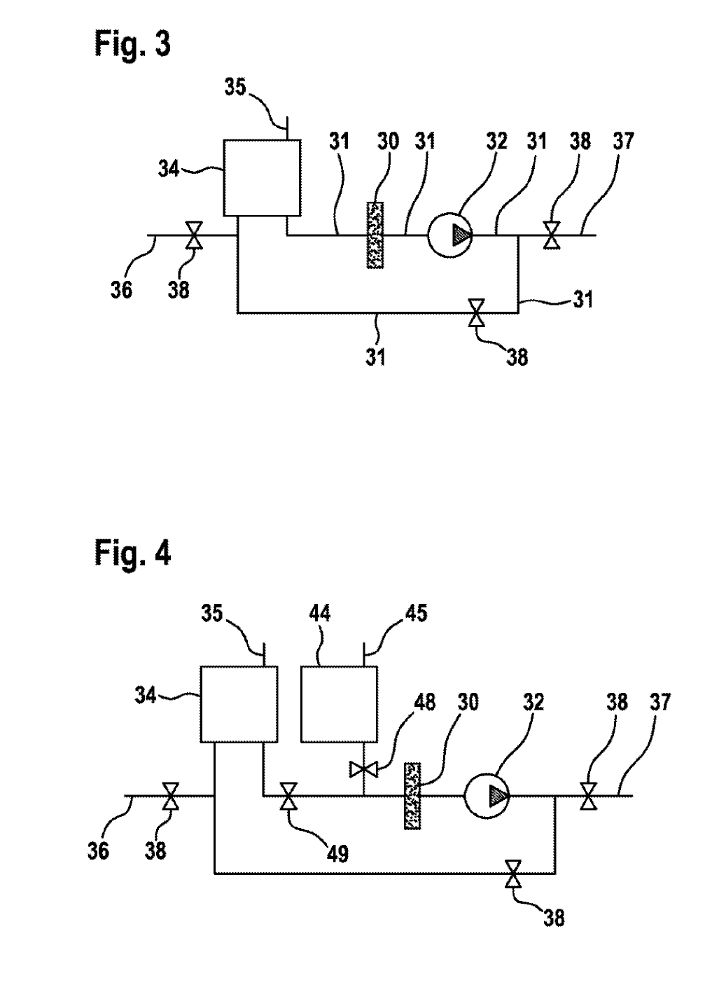

FIG. 3 shows a further variant of the device for carrying out the method. This design is especially suitable as a microfluidic system. In general, a microfluidic system has the advantage that the dead volume of the structure can be kept very low and the risk of foam formation is low. The circular fluid guidance in the fluidic connections 31 is driven by the pump 32. The filter chamber 30 is situated upstream of the pump. Furthermore, a vessel 34 is provided which can be vented via an opening or a venting channel 35. The opening or the venting channel 35 is advantageously situated at the upper end of the vessel with regard to gravity. As a result, an unintended leakage of reagents can be avoided. An inlet channel 36 is provided upstream of the vessel 34, it also being possible for the inlet channel 36 to open directly into the vessel 34. Furthermore, it is also possible for multiple inlet channels 36 to be present. An outlet channel 37 is situated downstream of the pump. The flow of the liquids is controlled via the integrated valves 38, which are arranged at various points in the system. In this case, the valves can, for example, be rotary valves or membrane valves.

When using such a device, the method according to the invention can be carried out as follows: The sample, i.e., the liquid containing the biological cells, is introduced into the vessel 34 via the inlet channel 36 or by introduction (e.g., pipetting) through the opening 35. The vessel 34 can, for example, be realized as a microfluidic cavity. The air present in the vessel is in this case released via the opening or the venting channel 35, and so the vessel 34 is vented. The pump 32 then pumps the sample across the filter 30 in the direction of the outlet channel 37. It can be envisaged that the venting channel 35 is closed, and so the pump 32 can suck the sample directly across the inlet channel 36. The cells present in the sample collect or accumulate on the filter 30. Thereafter, the cells are disrupted by, for example, being treated with an appropriate lysis buffer. The lysis buffer is first introduced into the vessel 34, for example via the inlet channel 36. Thereafter, the lysis buffer is pumped in a circuit by the pump 32 across the filter 30 via the circular fluidic path 31. Alternatively, the cell disruption can also proceed in a different way, for example by means of ultrasound. In this case, the filter 30 is appropriately ultrasonicated. A binding step then takes place in which the target molecules adsorb to the filter 30. To this end, an appropriate binding buffer is introduced into the vessel 34 and pumped in a circuit 31. For the following wash step, the wash buffer is first introduced into the vessel 34 and pumped by means of the pump 32 across the filter 30 in the direction of the outlet channel 37. When a drying of the filter 30 is intended, pumping of, for example, air or nitrogen is carried out from the inlet channel 36 across the filter 30 in the direction of the outlet channel 37. It is also possible to use the pump 32 for the drying. Finally, an elution step can take place, involving introducing an appropriate elution buffer into the vessel 34 and pumping it by means of the pump 32 across the filter 30 in the direction of the outlet channel.

In general, the introduction of the sample and of the buffers into the vessel 34 can, for example, be achieved by means of a further pump or manually by pipetting or the like. For this purpose, a reclosable opening can be provided in the vessel 34.

FIG. 4 shows a further variant of the system, with one or more further vessels 44, for example prestorage vessels, being provided in addition to the vessel 34. Said vessels 44 too are equipped with an opening or a venting channel 45. The content of the vessel 44 can be introduced into the rest of the conduit system via a further valve 48. Apart from that, the system substantially corresponds to the device depicted in FIG. 3. The corresponding elements are therefore given the same reference signs. A further valve 49 is provided between the vessel 34 and the feed line from the further vessel 44. It is possible to prestore various buffers in the vessel(s) 44, for example the lysis, digestion, denaturation, binding, wash or elution buffer. This variant has the advantage that an automatic performance is simplified, since the buffers no longer need to be introduced individually into the vessel 34. The method can be carried out such that especially the sample is introduced manually into the vessel 34 before being applied to the filter 30. The various required buffers in the subsequent process steps can be introduced in an automated manner from the vessel(s) 44.

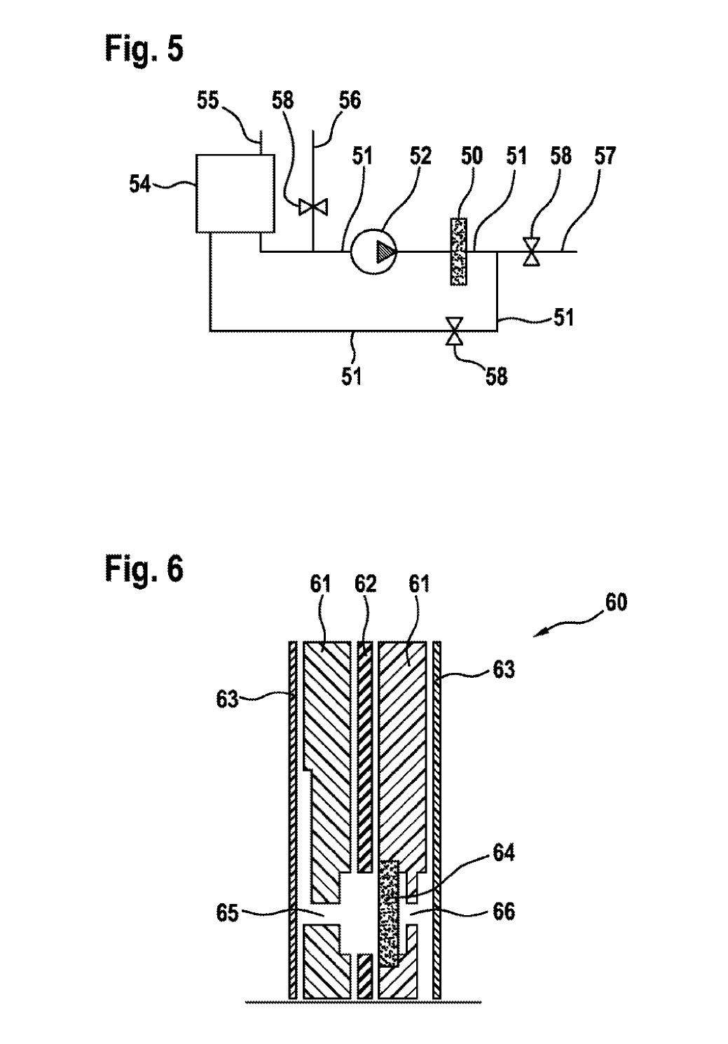

FIG. 5 illustrates a further preferred example of a device for carrying out the method according to the invention, which device can, for example, be realized in a microfluidic system. In this system, the pump 52 is arranged upstream of the filter chamber 50. This has the advantage that liquids can be pumped at a very high pressure across the filter 50. To this end, it is particularly advantageous when the channel piece or the tubing piece between the pump 52 and the filter 50 is comparatively short. The inlet channel 56 opens directly in front of the pump 52. This has the advantage that the liquids pumped from the inlet channel 56 across the filter 50, especially the sample containing the cell material, do not pass the prestorage vessel 54, and so contamination is avoided. Advantageously, parts or segments of the system can be heated, for example the filter 50 and/or the pump 52 and/or the vessel 54 can be heated. A heating can be appropriately carried out during the lysis step, making it possible for the cell disruption to proceed even more efficiently. An optimal temperature for the lysis enzymes used can be appropriately set, which temperature can, for example, be within a temperature range between 35 and 55° Celsius, for example at 45° Celsius. The process effectuation in the lysis step is preferably achieved in a circular manner via the circular fluidic path 51, comparable to the other exemplary embodiments described. Further inlet channels, via which the required reagents can be pumped into the system, can be present as variants. The vessel(s) 54 have, for example, a volume of 2 ml and the filter has, for example, a diameter between 2 and 10 mm. The fluid flow is controlled via the valves 58. The liquids can leave the system via the outlet channel 57.

An experimental procedure for the accumulation and lysis of cells and a purification of DNA in a microfluidic system according to the invention can, for example, be carried out as follows: 10⁵ Staphylococci in 1 ml of physiological saline solution are introduced into the system by pumping via the inlet channel 56 or by pipetting into the vessel 54 and are pumped by means of the pump 52 across the filter 50. For the lysis, 100 μl of lysis buffer are pipetted into the vessel 54 and pumped in a circuit by means of the pump 52 across the filter 50 and the channel system 51 for 10 minutes with simultaneous temperature adjustment to 45° Celsius. Thereafter, a digestion buffer and a binding buffer are successively pipetted into the vessel 54 and likewise circulated. This has the advantage that a very good mixing of the reagents takes place and nucleic acids are bound effectively to the filter 50. The filter 50 is subsequently washed by a wash buffer being pipetted into the vessel and pumped across the filter 50 into the outlet channel 57. The bound DNA is eluted with water by water being pipetted into the vessel 54 and pumped across the filter 50 into the outlet channel 57. The eluate is collected. In parallel, a reference is processed: 10⁵ Staphylococci in 1 ml of physiological saline solution are accumulated by centrifugation at 13 000 g and the supernatant is pipetted off. 100 μl of lysis buffer are added by pipetting, mixed, and incubated at 45° C. for 10 min. The resulting lysate is successively mixed with digestion buffer and binding buffer and applied to a commercially available column. Thereafter, the column is washed with wash buffer and the DNA eluted with 100 μl of water. An analysis of the samples is carried out by means of a quantitative PCR. The experimental results show that the method according to the invention can achieve comparable results as in the case of the reference, it being possible to achieve in the method according to the invention considerable labor savings owing to the possibilities of the simple automation.

FIG. 6 shows a section through an exemplary microfluidic filter chamber 60 based on a multilayer structure. Two structured polymer plates 61, a polymer membrane 62 lying therebetween, and externally overlying lidding films 63 form the multilayer structure. The filter 64 is inserted in an indentation in one of the polymer plates 61. The feeding of fluid and the removal of fluid are done via the inlet channel 65 and the outlet channel 66. The membrane 62 upstream of the filter 64 can exercise additional functionalities, for example a valve function. This structure is especially suitable for microfluidic designs.

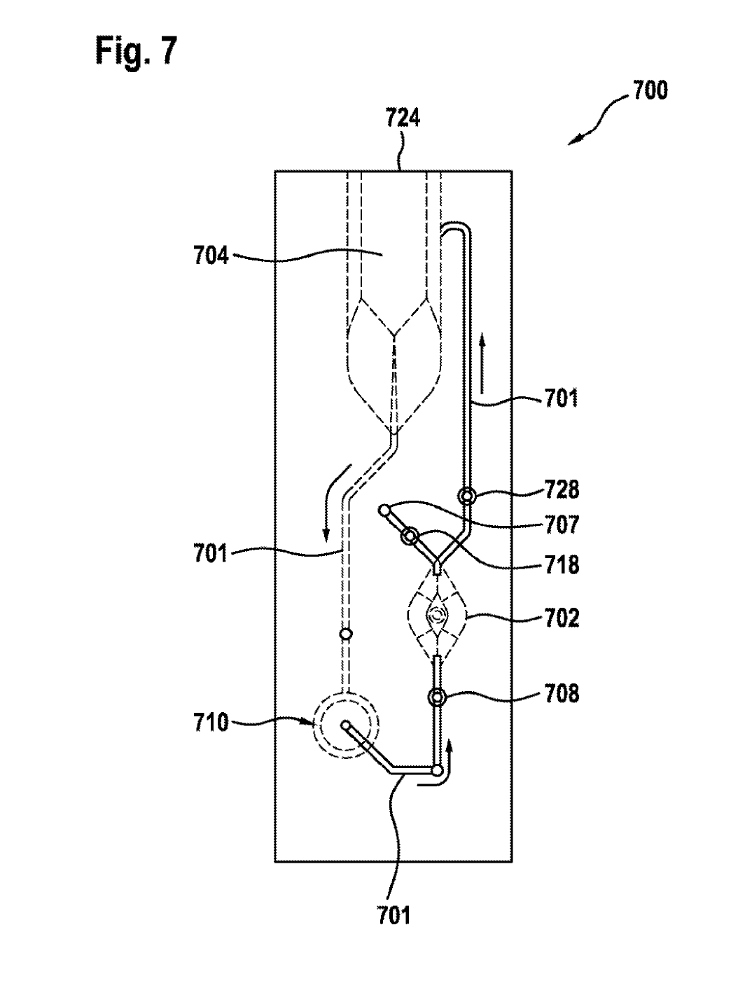

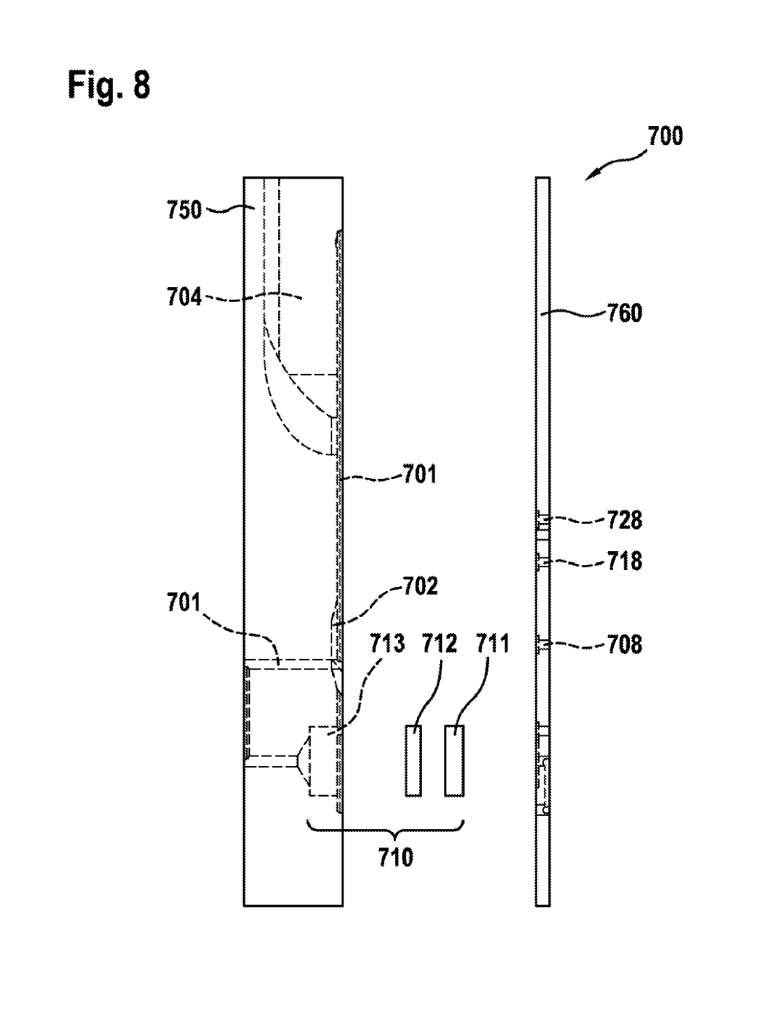

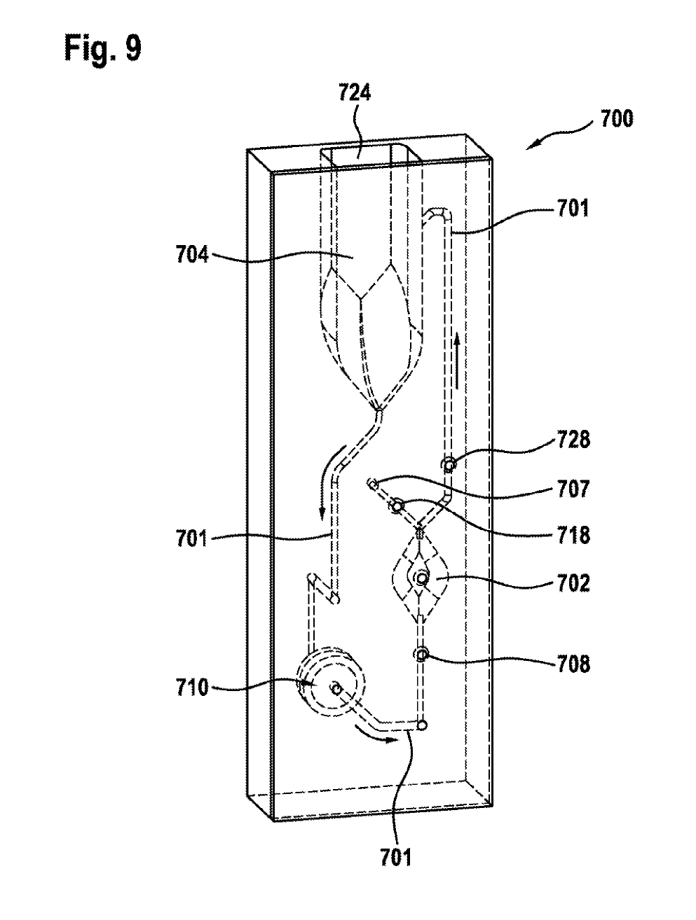

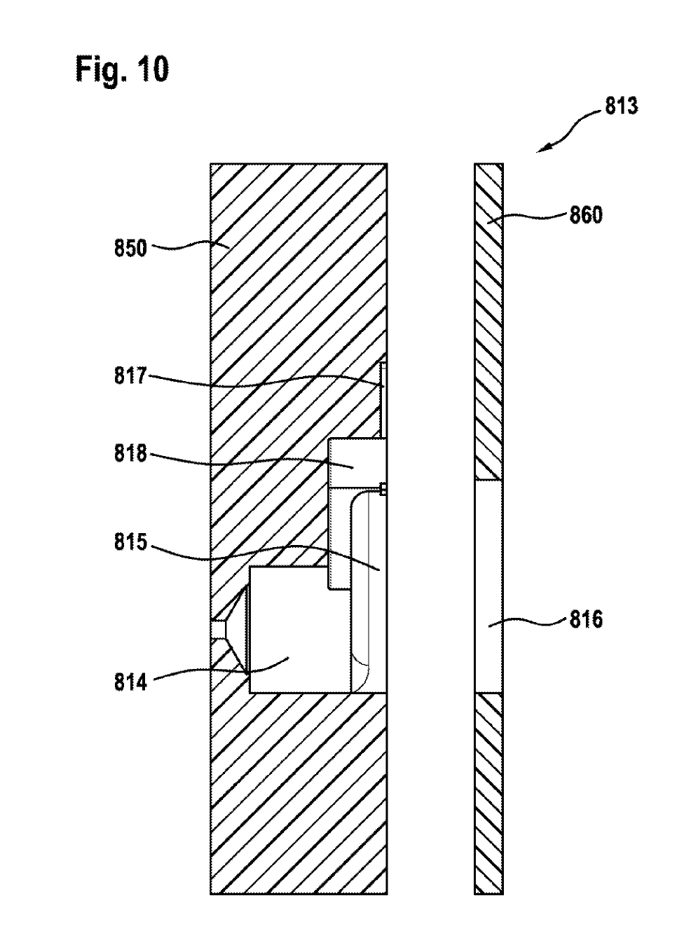

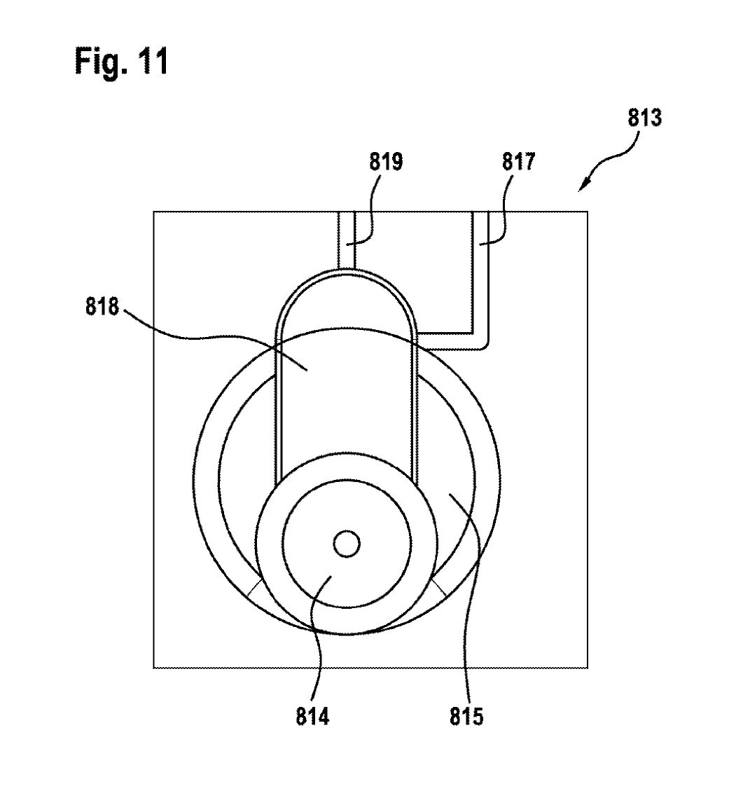

FIGS. 7 to 9 show an exemplary embodiment of a microfluidic system 700 for carrying out the method according to the invention. FIG. 7 shows a top view from the front, FIG. 8 shows a lateral view, and FIG. 9 shows a diagonal view from the front. The microfluidic system is realized as a multilayer structure composed of two structured polymer plates 750 and 760 (FIG. 8) and lidding films or other polymer layers (not depicted) for covering the structures, said lidding films being arranged on the right and left. The system comprises a pump 702, a vessel 704 having a vent opening 724, and a filter unit 710, and also multiple valves 708, 718, 728. Fluid guidance is carried out in the direction of the arrows (FIG. 7, FIG. 9), it also being possible to reverse the direction of flow. The filter unit 710 is formed by an indentation 713 (filter chamber) in the polymer plate 750, a frit 711 for the mechanical support of the filter, and the actual filter 712 (FIG. 8). Channels 701, which are covered by lidding films or other polymer layers (not depicted), run on the outer sides of the system. The valves 708, 718, 728 are realized as microfluidic membrane valves. The pump 702 is realized as a microfluidic membrane pump having a pumping chamber and an inlet valve 708 and two outlet valves 718, 728 and is downstream of the filter unit 710. The outlet valves 718, 728 form a T-junction and allow a switching of the fluid path between the circuit 701 (valve 728) and the outlet channel 707 (valve 718). An undepicted polymer membrane which is utilized for a pneumatic operation of the valves and of the pump is situated between the polymer plates 750 and 760. The vessel 704 is formed such that even the smallest quantities of liquid can run together at the lowest point of the vessel and, from there, enter into the channel system 701. To this end, the vessel 704 becomes narrower in a downward direction.

The depicted system 700 can be part of a larger microfluidic system which includes further functionalities, for example further pumps and mixing chambers, chambers for prestoring reagents, chambers for further processing of the biological molecules, for example for amplifying the nucleic acids obtained, for example by means of PCR, and components for detecting biological molecules, for example nucleic acids.

When using the microfluidic device 700, the method according to the invention can be carried out as follows: The sample is first introduced into the vented vessel 704, for example by pipetting and pumping, and then pumped from below across the filter unit 710 into the outlet channel 707. Then, lysis buffer is introduced into the vented vessel 704 and, during the lysis, circulated by means of the pump 702 across the filter unit 710. Alternatively, the lysis buffer can also be only briefly circulated in order to reliably fill the filter chamber 713 with liquid, and this is then followed by, for example, heat or ultrasound being applied to the filter 712. Thereafter, a binding buffer is added to the vented vessel 704 and circulated by means of the pump 702 across the filter unit 710. In the course of this, there is mixing of lysate and binding buffer and nucleic acids (as an example of molecules to be purified) bind to the filter 712. The mixture is then pumped into the outlet channel 707. Thereafter, a wash buffer is introduced into the vented vessel 704 and pumped across the filter unit 710 into the outlet channel 707. Lastly, an elution buffer is introduced into the vented vessel 704 and pumped across the filter unit 710 into the outlet channel 707. Alternatively, the elution buffer can also be sucked in via the outlet channel 707 and pumped in the reverse direction across the filter unit 710 into the vented vessel 704.

In one variant of this method, human cells present in the sample can first be selectively lysed. In a further variant of the method, a digestion of proteins can be carried out after the lysis. In a further variant of the method, a denaturation step can be carried out before the binding of the DNA. In a further variant of the method, the filter can be dried before the elution. In a further variant of the method, the pumping direction can, from time to time, be briefly reversed, for example for 5 to 60 s.

FIG. 10 shows a lateral view and FIG. 11 a top view of an exemplary embodiment of a filter chamber 813 for microfluidic devices. In FIG. 10, the multilayer structure composed of two polymer plates 850 und 860 is evident. The circular indentation 814 in the polymer plate 850 is provided as a blind hole for the insertion of a filter membrane or a filter material bed and possibly a frit. A circular expansion 815 is provided immediately above the filter to be inserted. In the other polymer plate 860, a circular aperture 816 is provided, which, in the mounted state, is covered by a lidding membrane (not depicted). A feeding or removal of liquids can be achieved via the channel 817. The circular aperture 816 can, for example, have a diameter between 5 and 50 mm and can be arranged more or less concentrically in relation to the filter, but also be displaced—especially upward with regard to the direction of gravity—for example by the difference between the radii of filter and circular area. Through the aperture 816 and the expansion 815, it is possible to input ultrasound by means of a sonotrode into the interior of the filter chamber 813, making it possible to carry out an ultrasound lysis. This variant has the advantage that an especially efficient ultrasound lysis is possible. Alternatively, the area intended for the input of ultrasound can also be oval, quadratic or elongated with comparable dimensions. Furthermore, this variant has the advantage that the expansion 815 of the filter chamber 813 can simultaneously perform the function that air bubbles rise upward in the expansion and are thus eliminated from the liquid circuit and that a liquid volume increasing during processing is accommodated, and can thus possibly replace another vessel of the system. For this purpose, an additional venting channel 819 can be provided. In this case, it is appropriate when the expansion has, for example, a volume between 500 μl and 5 ml. To this end, an additional expansion 818 can be provided. The filters used here can, for example, be silica membranes, but also beds of microbeads. In one variant, the filter or the bed of microbeads extends into the expansion 815 and the aperture 816, and so on this side there is contact with the lidding membrane. This has the advantage that, when coupling in ultrasound into the lidding membrane, the filter or the bed of microbeads is made to vibrate in an especially efficiently manner, and, as a result, the lysis of accumulated cells can take place with a greater yield.

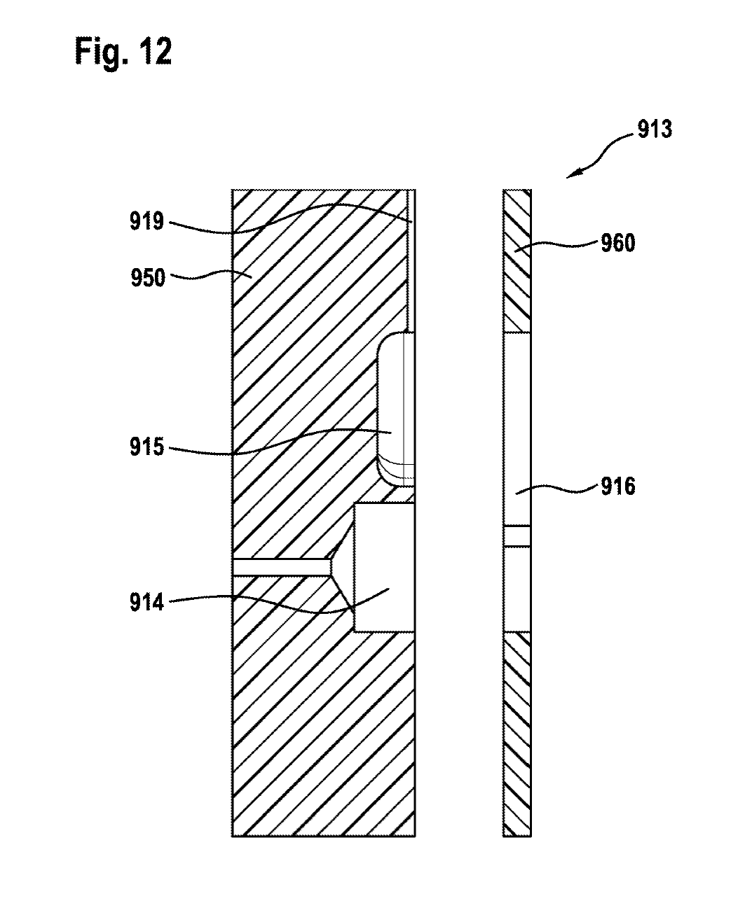

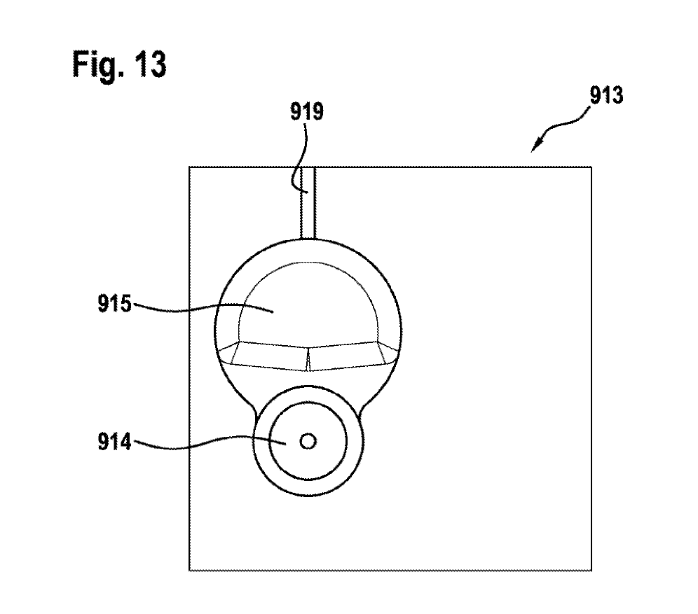

FIG. 12 and FIG. 13 show a further embodiment for a filter chamber 913 which, comparable with the filter chamber 813, has a blind-hole indentation 914 in the polymer plate 950 for the accommodation of a filter or a filter material bed and possibly a frit. A feeding or removal of liquids can take place via the channel 919. A part-circular expansion 915 of the blind hole 914 is provided in the polymer plate 950. The other polymer plate 960 has, in these areas, an aperture 916 which, in the mounted state, establishes the connection between the blind hole 914 and the expansion 915 and which is covered by a lidding membrane (not depicted). Comparable with the embodiment 813, the expansion 915 and the aperture 916 are likewise suitable for a coupling in of ultrasound.

1. A method for of purifying biological molecules, comprising:
pumping at least a portion of liquids to be used in an effectuation process into a circuit across the filter, the pumping including:
pumping liquid containing biological molecules across a filter, such that molecules retained on the filter are disrupted;
pumping a binding buffer configured to bind the biological molecules to the filter into the circuit across the filter to bind the molecules retained on the filter to the filter; and
pumping a wash buffer configured to clean the biological molecules bound to the filter across the filter.

2. The method as claimed in claim 1, further comprising:
pumping a lysis buffer configured to disrupt the molecules retained on the filter into the circuit across the filter; or
performing a mechanical disruption process to mechanically disrupt the molecules retained on the filter.

3. The method as claimed in claim 1, further comprising:
performing a denaturation process prior to the pumping of the binding buffer.

4. The method as claimed in claim 1, further comprising:
performing an additional digestion process prior to the pumping of the binding buffer.

5. The method as claimed in claim 1, further comprising:
drying the filter after the pumping of the wash buffer.

6. The method as claimed in claim 1, further comprising:
before the pumping of the liquid containing the biological cells across the filter, performing a pretreatment of the liquid.

7. The method as claimed in claim 1, wherein at least one of the pumpings is performed in conjunction with an input of heat.

8. The method as claimed in claim 1, wherein at least one of the pumpings includes reversing a pumping direction at least once.

9. The method as claimed in claim 1, wherein the method is carried out in a microfluidic device.

10. A device for carrying out a purification of biological molecules, comprising:
at least one filter;
a conduit system having at least one pump configured to circularly pump liquids across the at least one filter; and
a unit configured to fix the at least one filter.

11. The device as claimed in claim 10, wherein:
the unit has a body that defines a filter chamber with a multilayer structure, and
the filter chamber is configured to receive a filter membrane or a filter material.

12. The device as claimed in claim 11, wherein the filter chamber has includes:
an expansion that is configured to couple the at least one filter with a received ultrasound, and that has an aperture; and
a lidding membrane that closes off the aperture.

13. The device as claimed in claim 10, further comprising:
at least one vented vessel configured to introduce liquids into the system.

14. The device as claimed in claim 13, wherein at least one of the at least one filter, the pump, the at least one vessel, and the conduit system is operable to produce heat.

15. The device as claimed in claim 10, wherein the device is a microfluidic system.

16. The method of claim 2, wherein the mechanical disruption process includes an ultrasound treatment.

17. The method of claim 6, wherein the pretreatment process includes selectively lysing human cells present in the liquid.
